(19) United States
(12) Patent Application Publication
(10) Pub. No.: US 2007/0248653 A1
(43) Pub. Date: Oct. 25, 2007

(54) Hemostatic compositions and methods for controlling bleeding

(76) Inventors: Kent C. Cochrum, West Sacramento, CA (US); Susan Jemtrud, Auburn, CA (US)

Correspondence Address: FISH & RICHARDSON P.C., PO BOX 1022, MINNEAPOLIS, MN, 55440-1022, US

(21) Appl. No.: 11/407,459

(22) Filed: Apr. 20, 2006

(51) Int. Cl. A61K 31/717; A61L 15/00

(52) U.S. Cl. 424/445; 514/57

(57) ABSTRACT

The disclosure provides hemostatic compositions useful to promote hemostasis at active bleeding wound sites. The hemostatic compositions include an article containing cellulose, e.g., cotton gauze, and a cross-linked polysaccharide ionically linked to the cellulose. Methods of making and using the hemostatic compositions are also provided.

### TECHNICAL FIELD

This disclosure relates to hemostatic compositions and methods employing the same, and more particularly to hemostatic compositions useful for controlling bleeding at active bleeding wound sites.

### BACKGROUND

Wounds are generally classified as acute or chronic in accordance with their healing tendencies. Acute wounds, typically those received as a result of surgery or trauma, usually heal uneventfully within an expected time frame. Acute wounds include wounds such as active bleeding wound sites, e.g., wounds that have detectable, unclotted blood. The rapid control of topical bleeding at active bleeding wound sites is of critical importance in wound management, especially for the management of trauma, e.g., as a result of military exercises or surgery.

A conventional method of controlling bleeding at active bleeding wound sites, such as an external hemorrhage or a surgical wound, advocates the use of cotton gauze pads capable of absorbing 250 ml of blood. Cotton pads are considered passive, however, because of their inability to initiate or accelerate blood clotting. Other formulations have been reported to promote hemostasis and are described in U.S. Pat. Nos. 6,454,787; 6,060,461; 5,196,190; 5,667,501; 4,793,336; 5,679,372; 5,098,417; and 4,405,324. A hemostatic composition capable of accelerating the coagulation cascade to form a thrombus would be useful.

### SUMMARY

Accordingly, the disclosure provided herein relates to hemostatic compositions and methods for making and using the same in order to promote hemostasis at active bleeding wound sites. The present compositions typically include an article which contains cellulose, e.g., cotton gauze, and a crosslinked (e.g, covalently or ionically cross-linked) polysaccharide ionically linked to the cellulose.

In one aspect of the disclosure, a method for controlling bleeding at an active bleeding wound site of an animal is provided. The animal can be a mammal. For example, the animal can be a human, horse, bird, dog, cat, sheep, cow, or monkey. The method includes applying a hemostatic composition to the active bleeding wound site. Wound sites can include parenchynal organs (e.g., liver, kidney, spleen, pancreas, or lungs) or arteries and veins (e.g., pulmonary artery and vein, aorta, vena cava, carotid artery and jugular vein, subclavian artery and vein, axillary artery and vein, brachial artery and vein, thoracic artery and vein, radial artery and vein, ulnar artery and vein, illiac artery and vein, femoral artery and vein, popliteal artery and vein, or tibial artery and vein).

The hemostatic composition includes an article which contains cellulose and a cross-linked polysaccharide, such as covalently crosslinked dextran, alginate, or starch, or ionically cross-linked alginate (e.g., via Ca²⁺ ions), which is ionically linked to the cellulose. In some embodiments, a covalently crosslinked polyol such as covalently crosslinked polyvinyl alcohol, sorbitol, or polyvinyl pyrollidone can be ionically linked to the cellulose. A cross-linked polysaccharide may be porous, e.g., covalently crosslinked dextran beads. A cross-linked polysaccharide may be in a particle, bead or sphere form. For example, if covalently crosslinked dextran is used, it may be in the form of a bead, e.g., covalently crosslinked dextran beads. The molecular weight of dextran prior to crosslinking can range from about 10,000 to about 2,000,000 Daltons, or from about 20,000 to about 100,000 Daltons. In some embodiments, if covalently cross-linked starch is used, it may be in the form of starch microspheres, such as degradable starch microspheres (DSM).

When a crosslinked polysaccharide is ionically linked to the cellulose, it can have a molecular weight exclusion limit of greater than about 10,000 Daltons when dry. When fully hydrated, the molecular weight exclusion limit ranges from greater than 30,000 Daltons to greater than 300,000 Daltons (e.g., greater than 70k, 100K, 150K, 300K, 450K, and 600K).

Articles which contain cellulose can be barriers, structures, or devices useful in surgery, diagnostic procedures, or wound treatment. For example, an article containing cellulose can be a bandage, suture, dressing, gauze, gel, foam, web, film, tape, or patch. An article containing cellulose can include a cotton material, e.g., cotton gauze or lap sponge. In other embodiments, the article containing cellulose can be synthetic gauze (e.g., rayon/polyester), oxidized regenerated cellulose, or spot applicator such as a modified Q-Tip®. The article can also optionally include adhesives or polymeric laminating materials.

The article containing cellulose can be used singularly or combined as needed to properly treat a wound site. For example, one piece of cotton gauze with dimensions of about 10 cm×10 cm can be treated with a polysaccharide and a solution of saline to ionically link the polysaccharide to the cellulose. These sheets may then be assembled and used together to provide proper wound coverage and initiate hemostasis.

Hemostatic compositions of the present disclosure are useful for accelerating blood clotting at an active bleeding wound site. Prior to the application of a hemostatic composition, an active bleeding wound site may be characterized in that it bleeds at a rate of from about 0.5 ml/min to about 1000 ml/min, for example, 0.5 ml/min to 500 ml/min, 0.5 ml/min to 200 ml/min, 0.5 to 100 ml/min, 0.5 ml/min to 25 ml/min, 1 ml/min to 10 ml/min, 1 ml/min to 100 ml/min, 1 ml/min to 500 ml/min, 10 ml/min to 100 ml/min, 10 ml/min to 250 ml/min, 10 ml/min to 500 ml/min, 10 ml/min to 1000 ml/min, 50 ml/min to 250 ml/min, or 50 ml/min to 500 ml/min. After application of a hemostatic composition, the active bleeding wound site may bleed at a rate of less than 0.03 ml/min., for example, the rate of less than 0.03 ml/min. may be achieved in from about 2 to about 20 minutes, and in certain embodiments in less than about 5 minutes.

In neurological, opthalmic, or spinal embodiments, where even the smallest amount of blood flow can have a substantial effect on the patient, an active bleeding site may be characterized by a rate of blood flow from 0.1 ml/min to 20 ml/min, for example, 0.1 ml/min to 10 ml/min, 0.1 ml/min to 5 ml/min, 0.1 ml/min to 1 ml/min, 0.1 ml/min to 0.5 ml/min, 0.25 ml/min to 20 ml/min, 0.25 ml/min to 10 ml/min, 0.25 ml/min to 5 ml/min, 0.25 ml/min to 1 ml/min, 0.25 ml/min to 0.5 ml/min.

In certain embodiments, some of a cross-linked polysaccharide may also be physically trapped in fibers of the article comprising cellulose.

In further embodiments, hemostatic compositions are provided that include additional agents, such as analgesics, steroids, antihistamines, anesthetics, bactericides, disinfectants, fungicides, vasoconstrictors, hemostatics, chemotherapeutic drugs, antibiotics, keratolytics, cauterizing agents, antiviral drugs, epidermal growth factor, fibroblast growth factors, transforming growth factors, glycoproteins, collagen, fibrinogen, fibrin, thrombin, humectants, preservatives, lymphokines, cytokines, odor controlling materials, vitamins, and clotting factors.

The disclosure also provides methods for making hemostatic compositions. Hemostatic compositions of the present disclosure can be made by contacting (e.g., spraying, wetting, covering, or coating) an article comprising cellulose with a solution comprising a cation, followed by contacting (e.g., spraying, coating, applying, sprinkling, covering, or dusting) the cellulose with a cross-linked polysaccharide to form a hemostatic composition having the cross-linked polysaccharide ionically linked to the cellulose. The ionic linking occurs through available groups on the cross-linked polysaccharide to available groups on the cellulose via a cation linking agent. The cation can be any metal cation, including K⁺; Na⁺; Li⁺; Mg²⁺; Ca²⁺; Ba²⁺; Zn²⁺; Cu²⁺; Fe³⁺; and Al³⁺. In certain embodiments the cation is Na⁺, which may be in the form of, or derived from, a solution of sodium chloride in water. For example, the hydroxyl groups on cross-linked dextran may be linked to the hydroxyl groups on cellulose via a Na⁺ ion.

The cation linking agent may be delivered in the form of an aqueous solution. This solution comprises a cation and an anion dissolved in a solvent, e.g., water. The cation may be as described previously, for example, Na⁺. The anion can be F⁻, Cl⁻, Br⁻, I⁻, SO₄²⁻, PO₃³⁻, C₂H₃O⁻, C₆H₅O₇²⁻, C₄H₄O₆²⁻, C₂O₄²⁻, HCOO⁻, BO₃³⁻, and CO₃²⁻. For example, in some embodiments, a 0.9% solution of sodium chloride is sprayed onto the surface of cellulose and dusted with 2 g of crosslinked dextran. An additional advantage to the use of sodium chloride is its known antiseptic qualities. For example, the dried compositions may have a high local concentration of sodium chloride, which may be capable of inhibiting microbial growth.

In certain embodiments of the method, the cross-linked polysaccharide is covalently cross-linked dextran. The cross-linked dextran can be in the form of covalently cross-linked beads, which may be porous. The molecular weight of the dextran prior to crosslinking can range from about 10,000 to about 2M, or from about 20,000 to about 100,000 Daltons.

Unless otherwise defined, all technical and scientific terms used herein have the same meaning as commonly understood by one of ordinary skill in the art to which this disclosure belongs. Although methods and materials similar or equivalent to those described herein can be used to practice that which is set out in this disclosure, suitable methods and materials are described below. All publications, patents, patent applications, and other references mentioned herein are incorporated by reference in their entirety. In case of conflict, the present specification, including definitions, will control. In addition, the materials, methods, and examples are illustrative only and not meant to be limiting.

The details of one or more embodiments of the disclosure are set forth in the accompanying drawings and the description below. Other features, objects, and advantages of the disclosure will be apparent from the description and drawings, and from the claims.

### DETAILED DESCRIPTION

As used herein, the terms “linking” or “linked” are meant to indicate an ionic link, either direct or mediated by a chemical moiety such as an ion, between two chemically distinct entities, e.g., cross-linked dextran ionically linked to cellulose. The term “cross-link” is meant to indicate a covalent or ionic linkage, either direct or mediated by a chemical moiety or ion, between two chemically similar moieties, e.g., dextran covalently cross-linked to itself; alginate ionically cross-linked to itself. The chemically similar moieties do not have to be identical. For example, dextran having a particular average molecular weight range includes dextran molecules of a variety of molecular weights, and thus the dextran molecules are not identical but chemically similar. When dextran molecules having an average molecular weight range are linked, e.g., covalently linked with epichlorohydrin, they are said to be “cross-linked.”

The terms “spheres,” “particles,” or “beads,” when used in the context of the present disclosure, are not meant to imply different relative sizes among the terms, but are meant to be interchangeable terms describing an embodiment of a composition.

The term “active bleeding wound site” means, at a minimum, that unclotted blood is present in the wound, e.g., extravascular blood, particularly where the surface of a tissue has been broken or an artery, vein, or capillary system has been compromised. The rate of blood flow from an active bleeding wound site can vary, depending upon the nature of the wound. In some cases, an active bleeding wound site will exhibit blood flow at a rate from about at a rate of from about 0.5 ml/min to about 1000 m/min. Some active bleeding wound sites may exhibit higher rates of blood flow, e.g., punctures of major arteries such as the aorta. After application of the hemostatic composition, the active bleeding wound site may bleed at a rate of less than 0.03 ml/min. For example, the rate of less than 0.03 ml/min. may be achieved in from about 2 to about 20 minutes, and in certain embodiments in less than about 5 minutes.

Hemostatic Compositions

The disclosure provided herein relates to hemostatic compositions used to promote hemostasis at active bleeding wound sites. While not being bound by any theory, it is believed that the hemostatic compositions of the present invention control bleeding by initiating and accelerating blood clotting. The hemostatic compositions of the present disclosure activate platelets and concentrate high molecular weight components of the coagulation cascade (e.g., clotting factors) by excluding high molecular weight components of the cascade, while absorbing the lower molecular weight components in blood. Accordingly, coagulation cascade components having a molecular weight higher than about 30,000 Daltons are excluded, including fibrinogen (MW 340,000); prothrombin (MW 70,000); thrombin (MW 34,000); Factor V (MW 330,000); Factor VII (MW 50,000); Factor VIII (MW 320,000); von Willebrand factor (MW >850,000); Factor IX (MW 57,000); Factor X (MW 59,000); Factor XI (MW 143,000); Factor XII (MW 76,000); Factor XIII (MW 320,000); high MW kininogen (Fitzgerald Factor) (MW 120,000-200,000), and prekallikrein (Fletcher Factor) (MW 85,000-100,000). In addition, laboratory experiments indicate that platelets aggregate around the hemostatic compositions of the present disclosure when exposed to blood. The net result is that concentrated clotting factors (coagulation cascade components) and activated platelets accelerate the conversion of prothrombin to thrombin in the presence of Ca²⁺, which subsequently catalyzes the conversion of fibrinogen to insoluble fibrin multimers, e.g., a fibrin clot. Additional information on the clotting cascade and hemostatic compositions containing fibrin can be found in U.S. Pat. No. 5,773,033.

Hemostatic compositions typically include an article comprising cellulose, e.g., cotton gauze or a lap sponge, and a cross-linked polysaccharide ionically linked to the cellulose. The cross-linked polysaccharide may be ionically or covalently cross-linked. The cross-linked polysaccharide may be porous. The cross-linked polysaccharide may be in the form of beads, particles, or spheres.

Any suitable polysaccharide can be used; however, the polysaccharide chosen should typically be safe for in vivo use, e.g., non-allergenic and non-toxic. Suitable polysaccharides for clinical use are known in the art and available from a variety of sources. See, e.g., U.S. Pat. No. 5,837,547. In certain embodiments, a cross-linked polysaccharide can be covalently cross-linked dextran, starch, or alginate, or ionically cross-linked alginate. For example, covalently cross-linked dextran (e.g., in the form of beads can be used), or covalently cross-linked starch (e.g., potato starch, amylase, amylopectin, or mixtures thereof) can be used. Ionically cross-linked alginate can be used in some embodiments. Covalently cross-linked starch can be in the form of degradable starch microspheres (DSM). Details of the preparation of these spheres is detailed in U.S. Pat. No. 4,126,669, Example 1 or U.S. Pat. No. 4,124,705.

The average molecular weight range of the polysaccharide, typically measured before cross-linking, can vary, but can range from about 10,000 to about 2M Daltons. The molecular weight range chosen will affect the molecular weight exclusion limit of the ionically linked cross-linked polysaccharide, and thus its ability to exclude the coagulation components and concentrate them.

In some embodiments, covalently cross-linked dextran is preferred. Dextran is a high molecular weight polysaccharide that is water-soluble. It is non-toxic and tolerated well by most animals, including humans. The average molecular weight of dextran used in the present disclosure before cross-linking can range from about 10,000 to about 2,000,000 Daltons, or from about 20,000 to about 100,000 Daltons.

Covalently cross-linked dextran can be in the form of beads, e.g., covalently cross-linked beads, before it is linked ionically to the cellulose. Covalently cross-linked dextran can be porous. Covalently cross-linked dextran beads can exhibit a range of sizes, e.g., from about 30 to about 500 μm and molecular weight exclusion limits, e.g., from 1.5K to 600K. Covalently cross-linked dextran beads are commercially available, e.g., as Sephadex™ (Pharmacia); see, for example UK 974,054 or U.S. Pat. No. 3,042,667.

In other embodiments, hemostatic compositions of the present disclosure can include an article containing cellulose ionically linked to an ionically cross-linked polysaccharide, such as alginate. Ionic cross-linkages include ion-mediated bonds between available chemical moieties on the polysaccharide molecules. Typical chemical moieties that can be mediated with an ion (e.g., a cation) include hydroxyl moieties. For example, sodium alginate or alginic acid salts can be ionically cross-linked with metal cations, including Mg²⁺; Ni²⁺; Ca²⁺; Ba²⁺; Zn²⁺; Cu²⁺; Fe³⁺; and Al³⁺. Typically, Ca²⁺ may be used. Ionic linkages from the ionically cross-linked polysaccharide to cellulose can employ similar cations or those described previously.

The average molecular weight of the polysaccharide, the degree of ionic linking of the cross-linked polysaccharide to cellulose, and the degree of cross-linking of the polysaccharide to itself are factors in the molecular weight exclusion limit of the polysaccharide in a hemostatic composition and the water regain of a hemostatic composition. Water regain is defined as the weight of water taken up by 1 g of dry hemostatic composition and can be determined by methods known in the art. For example, it is known that small changes in dextran concentration or cross-linking agent concentration (e.g., epichlorohydrin) can result in dramatic changes in water regain. Typically, at lower molecular weights of dextran, a higher water regain results. See Flodin, P., “Chapter 2: The Preparation of Dextran Gels,” Dextran Gels and Their Applications in Gel Filtration, Pharmacia, Uppsala Sweden, 1962, pages 14-26.

Similarly, the degree of hydration of the cross-linked polysaccharide also affects the molecular weight exclusion limit. As the degree of hydration increases, the molecular weight exclusion limit of the cross-linked polysaccharide usually increases. Typically, when covalently cross-linked dextran is ionically linked to cellulose, when dry, the covalently cross-linked dextran will have a molecular weight exclusion limit of greater than about 10,000 Daltons. When hydrated, the covalently cross-linked dextran can have a molecular exclusion limit of greater than 30,000 Daltons.

The article may include natural or synthetic celluloses (e.g., cellulose acetate, cellulose butyrate, cellulose propionate, oxidized regenerated cellulose). In some embodiments, the article comprising cellulose may include synthetic gauze (e.g., rayon/polyester), or oxidized regenerated cellulose. These additional sources of cellulose are commercially available, e.g., as Surgicel® (Johnson & Johnson); see, for example, US 2004/0101546.

As used herein, ionic linkages encompass bonds from any of the available chemical moieties of the cross-linked polysaccharide to any of the available chemical moieties of the cellulose linked via a cation. The cation can be K⁺; Na⁺; Li⁺; Mg²⁺; Ca²⁺; Ba²⁺; Zn²⁺; Cu²⁺; Fe³⁺; and Al³⁺. For example, if covalently cross-linked dextran is used, available hydroxyl moieties on the dextran can be ionically linked to available hydroxyl moieties on the cellulose through the linking agent Na⁺.

The cation used as a linking agent to link the polysaccharide to the cellulose may be delivered in the form of an aqueous solution. This solution will comprise a cation and an anion dissolved in a solvent, e.g., water or a buffer. The cation may be as described previously, for example, Na⁺. The anion can be F⁻, Cl⁻, Br⁻, I⁻, SO₄²⁻, PO₃³⁻, C₂H₃O⁻, C₆H₅O₇²⁻, C₄H₄O₆^{2-, C}₂O₄²⁻, HCOO⁻, BO₃³⁻, and CO₃²⁻. For example, a solution of sodium chloride can be sprayed onto a surface of cellulose in a concentration from about 0.1% to about 3% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3%), or from about 0.5% to about 1.5%. In some embodiments, the article of cellulose will be treated with a solution comprising 0.9% sodium chloride. 14004-021001

Articles which contain cellulose can be any barriers, structures, or devices useful in surgery, diagnostic procedures, or wound treatment. For example, an article containing cellulose can be a bandage, suture, dressing, gauze, gel, foam, web, film, tape, or patch. An article containing cellulose can include a cotton material, e.g., cotton gauze. The article should allow the polysaccharide linked to the cellulose to interact with the wound site. The article containing cellulose can be used singularly or combined as needed to properly treat a wound site. For example, a piece of 16-ply cotton gauze with dimensions of about 10 cm×10 cm can be treated with a polysaccharide and a solution of saline to ionically link the polysaccharide to the cellulose. These sheets may then be assembled and used together to provide proper wound coverage and initiate hemostasis.

Hemostatic compositions can include additional agents, such as analgesics, steroids, antihistamines, anesthetics, bactericides, disinfectants, fungicides, vasoconstrictors, hemostatics, chemotherapeutic drugs, antibiotics, keratolytics, cauterizing agents, antiviral drugs, epidermal growth factor, fibroblast growth factors, transforming growth factors, glycoproteins, collagen, fibrinogen, fibrin, thrombin, humectants, preservatives, lymphokines, cytokines, odor controlling materials, vitamins, and clotting factors. For further information on these additional agents for incorporation, refer to WO 00/27327.

Hemostatic compositions may be used in combination with polymeric laminating materials and adhesives to provide both mechanical support and flexibility to an article and to facilitate adhesion to the wound. Additional information on such polymeric laminating materials and adhesives for use in the present disclosure can be found in, e.g., WO 00/27327.

Methods of Controlling Bleeding

In one aspect of the disclosure, a method for controlling bleeding at an active bleeding wound site of an animal is provided. The method includes applying a hemostatic composition to the active bleeding wound site. Application of the hemostatic composition typically includes contacting the hemostatic composition with the wound or bleeding site surface. The hemostatic composition is maintained in contact with the wound or bleeding site for a period of time sufficient to control the bleeding, e.g., to clot the blood, slow the rate of bleeding, or stop the bleeding. The application may include the use of pressure, e.g., by using an elastic bandage to maintain contact with the bleeding site. Alternatively, an internal wound may be packed with a hemostatic composition until hemostasis is achieved.

Usually a hemostatic composition can control bleeding, for example, to a rate of less than 0.03 ml/min, in a period of from about 2 to about 20 minutes. In certain embodiments, bleeding stops immediately, or in less than about 5 minutes.

Typically a hemostatic composition of the present disclosure will be used to inhibit or completely stop bleeding at or in an organ, such as the liver, kidney, spleen, pancreas, or lungs; or to control bleeding during surgery (e.g., abdominal, vascular, gynecological, dental, tissue transplantation surgery, etc.). For example, percutaneous needle biopsies are common interventional medical procedures. Possible complications of needle biopsies, however, include bleeding at the biopsy site. The amount of bleeding is related to the needle size, tissue sample size, location of the biopsy, and vascularization of the tissue. Hemostatic compositions of the present disclosure can be used to promote hemostasis at needle biopsy sites. For more information on biopsy tracts, see U.S. Pat. No. 6,447,534.

Another application of the hemostatic compositions provided herein will be to impede or halt completely bleeding at the site of an arterial or venous wound, such as the femoral, carotid, jugular, aorta, vena cava, or pulmonary arteries or veins, which may be the result of an injury incurred while performing military exercises. For example, the incidence of injuries to the lower extremities is high in modern warfare, and the majority of deaths which result from these injuries stem from wounds to the femoral artery. The hemorrhaging which occurs from wounds occurring at the femoral artery is often uncontrollable under field conditions and may result in the necessity of limb amputation or death. The hemostatic compositions described in this disclosure may offer a method of field hemostasis which may assist in lessening the complications resulting from these types of injuries.

The amount of hemostatic composition to be used will vary with the patient, the wound, and the composition employed. For example, hemostatic compositions with varying water regains can be assembled (e.g., stacked in descending order) for use in major bleeding to attain hemostasis.

Methods for Making Hemostatic Compositions

In another aspect, the present disclosure provides methods for making hemostatic compositions. The hemostatic compositions of the present invention can be made by applying a solution comprising a cation to an article containing cellulose, such as by spraying, coating, sprinkling, etc., followed by application (e.g., by dusting, spraying, sprinkling, coating, covering, scattering) of a cross-linked polysaccharide to form a hemostatic composition having the cross-linked polysaccharide ionically linked to the cellulose.

Any biologically compatible bifunctional or heterobifunctional reagent can be used as a covalent cross-linking agent, including reagents with halogens, epoxides, hydroxy succinimide esters, aldehydes, activated thiols, or other moieties for reacting free amines, hydroxides, hydroxyls, or sulfhydryls on the polysaccharide. A polysaccharide may also be modified, e.g., derivatized with suitable moieties, to facilitate such cross-linking, provided that the polysaccharide so derivatized remains pharmaceutically suitable for animal, e.g., human use. For additional information, see Flodin, P., and Ingelman, B., “Process for the Manufacture of Hydrophilic High Molecular Weight Substances,” British Patent No. 854, 715; and Flodin, P., “Chapter 2: The Preparation of Dextran Gels,” Dextran Gels and Their Applications in Gel Filtration, Pharmacia, Uppsala Sweden, 1962, pages 14-26.

An ionic linking agent for linkage to the article comprising cellulose may be, for example, sodium chloride, calcium chloride, sodium bicarbonate, or potassium phosphate.

In certain embodiments of the method, the crosslinked polysaccharide is covalently cross-linked dextran. The cross-linked dextran can be in the form of covalently cross-linked beads. The molecular weight of the dextran prior to crosslinking can range from about 10,000 to about 2M, or from about 20,000 to about 100,000 Daltons. Typically, dextran of MW 40,000 is used. The crosslinked polysaccharide may be applied to an article of cellulose which has been treated with a solution of a cation (e.g., a solution of Na⁺) in amounts ranging from 1×10⁻⁴ g/cm² of cellulose to about 3×10⁻³ g/cm² (e.g., 1×10⁻⁴ g/cm², 1.5×10⁻⁴ g/cm², 2×10⁻⁴ g/cm², 2.5×10⁻⁴ g/cm², 3×10⁻⁴ g/cm², 3.5×10⁻⁴ g/cm², 4×10⁻⁴ g/cm², 4.5×10⁻⁴ g/cm², 5×10⁴ g/cm², 5.5×10⁴ g/cm², 6×10⁻⁴ g/cm², 6.5×10⁻⁴ g/cm², 7×10⁻⁴ g/cm², 7.5×10⁻⁴ g/cm², 8×10⁻⁴ g/cm², 8.5×10⁻⁴ g/cm², 9×10⁻⁴ g/cm², 9.5×10⁻⁴ g/cm², 1×10⁻³ g/cm², 1.5×10⁻³ g/cm², 2×10⁻³ g/cm², 2.5×10⁻³ g/cm², 3×10⁻³ g/cm²) or from about 1×10⁻³ g/cm² to about 2×10⁻³ g/cm².

In another aspect, the disclosure provides a method of making a hemostatic composition including incubating an ionically cross-linked polysaccharide and a cation with an article containing cellulose in order to form a hemostatic composition having the article containing cellulose ionically linked with the ionically cross-linked polysaccharide (e.g., ionically cross-linked alginate with Ca²⁺). The cation which ionically links the ionically crosslinked polysaccharide to the cellulose may be as described previously, including, for example, Na⁺. The Na⁺ may be in the form of, or derived from, a saline solution.

A number of embodiments of the disclosure have been described. Nevertheless, it will be understood that various modifications may be made without departing from the spirit and scope of the disclosure. Accordingly, other embodiments are within the scope of the following claims.

### EXAMPLES

### Example 1

A 4 in.×4 in. (10.2 cm×10.2 cm) pad of 16-ply cotton gauze was unfolded to 4 in.×16 in. (10.2 cm×40.8 cm). 2 ml of 0.9% saline (3.08×10⁻⁴ mols NaCl) was sprayed on each unfolded gauze with a mister. Care was taken to ensure that the solution was sprayed directly onto the gauze. 2 g of Sephadex G-100 was dusted uniformly over the gauze. The gauze/saline/Sephadex composition was allowed to sit at room temperature for 60 minutes and dried at 55° C. for 48 hours.

### Example 2

A 10.2 cm×10.2 cm×0.65 cm (4 in.×4 in.×0.25 in.) piece of Surgicel® Fibrillar absorbable hemostat was cut into sections. One 10.2 cm×10.2 cm×0.16 cm section (4 in.×4 in.×0.06 in.) was sprayed with 0.35 ml of 0.9% saline. 0.24 g of Sephadex G-100 was dusted over the section. The Surgicel®/saline/Sephadex composition was allowed to dry at room temperature.

### Example 3

A porcine spleen incision model was used to evaluate the hemostatic capabilities of the compositions of Examples 1 and 2. A linear incision 3 cm in length and 0.4 cm in depth was made in the spleen with a surgical blade for each composition to be tested. Each incision was allowed to bleed for 30 seconds before applying the composition with mild pressure. Mild pressure was applied for 2 minutes before being released to observe for evidence of bleeding. Thereafter, pressure alone or more bandages accompanied by pressure was applied at one minute intervals as necessary. Hemostasis was called at the earliest time at which pressure was released without further bleeding into the gauze or leaking beyond the edges of the gauze onto the spleen up to a total of 11 minutes.

Bleeding rate was determined visually and assigned a value, e.g., a value of +2 corresponds to a bleeding rate of 1-2 ml/min and an assignment of +3 corresponds to a bleeding rate of 3-6 ml/min. The results, shown in Table 1, demonstrate that gauze/saline/Sephadex compositions were able to stop bleeding in all nine sites with an average time of 3.3 minutes to hemostasis using 1 gauze per site. Plain gauze was only able to achieve hemostasis in five of nine sites within 11 minutes, with an average time to hemostasis of >7.4 minutes using an average of 3.4 gauzes per site.

The Surgicel®/saline/Sephadex composition was able to stop bleeding in 3.5 minutes.

The results indicated that compositions having 0.125 g of Sephadex G-100 per in² (0.019 g per cm²) of matrix were effective in inducing rapid hemostasis.

The experiments were repeated using a pig femoral artery model. The results were similar to those obtained with the spleen incision model in that all gauze/saline/Sephadex hemostatic compositions achieved hemostasis within 11 minutes.

TABLE 1
Hemostatic capabilities of gauze compositions.

| | | | |
|---|---|---|---|
| Pig | Degree of Bleeding | Time to Stop (min) | Bandages Used |

Plain Gauze

| | | | |
|---|---|---|---|
| 1 | +2 | 3.0 | 2 |
| 2 | +2 | >11 | 2 |
| | +2 | >11 | 2 |
| 3 | +2 | 4.0 | 3 |
| | +2 | 5.0 | 4 |
| | +3 | >13 | 6 |
| | +2 | 2.5 | 3 |
| 4 | +2 | 3.3 | 5 |
| | +2 | >11 | 4 |
| Total | 19 | >66.3 | 31 |
| Average | 2.1 | >7.4 | 3.4 |

Saline/Sephadex/Gauze Compositions

| | | | |
|---|---|---|---|
| 1 | +2 | 2.0 | 1 |
| | +2 | 2.0 | 1 |
| 2 | +2 | 2.0 | 1 |
| | +3 | 8.0 | 1 |
| 3 | +2 | 2.0 | 1 |
| | +2 | 2.0 | 1 |
| | +3 | 5.0 | 1 |
| 4 | +2 | 3.0 | 1 |
| | +3 | 4.0 | 1 |
| Total | 21 | 30 | 9 |
| Average | 2.3 | 3.3 | 1 |

### Example 4

4 in.×4 in. (10.2 cm×10.2 cm) pads of 16-ply cotton gauze were unfolded to 4 in.×16 in. (10.2 cm×40.8 cm). From 0.5 ml to 5 mls of 0.9% saline (7.69×10⁻⁵ mols-7.69×10⁻⁴ mols NaCl) was sprayed on the unfolded gauzes with a mister. From 0.5 to 4.0 g of Sephadex G-100 was dusted uniformly over the gauzes. The gauze/saline/Sephadex composition was allowed to sit at room temperature for 60 minutes and dried at 55° C. for 48 hours.

### Example 5

A 4 in.×4 in. (10.2 cm×10.2 cm) pad of 16-ply cotton gauze was unfolded to 4 in.×16 in. (10.2 cm×40.8 cm). 2 ml of 0.9% saline (3.08×10⁻⁴ mols NaCl) was sprayed on the unfolded gauze with a mister. Care was taken to ensure that the solution was sprayed directly onto the gauze. 2 g of Degradable Starch Microspheres (DSM) were dusted uniformly over the gauze. The gauze/saline/DSM composition was allowed to sit at room temperature for 60 minutes and dried at 55° C. for 48 hours.

### P Example 6

A 10.2 cm×10.2 cm×0.65 cm (4 in.×4 in.×0.25 in.) piece of Surgicel® Fibrillar absorbable hemostat was cut into sections. One 10.2 cm×10.2 cm×0.13 cm section (4 in.×4 in.×0.05 in.) was sprayed with 0.5 ml of 0.9% saline. 0.5 g of Sephadex G-100 was dusted over the section. The Surgicel®/saline/Sephadex composition was allowed to dry at room temperature overnight before drying at 55° C. for 48 hours.

### Example 7

A 10.2 cm×10.2 cm×0.65 cm (4 in.×4 in.×0.25 in.) piece of Surgicel® Fibrillar absorbable hemostat was cut into sections. One 10.2 cm×10.2 cm×0.13 cm section (4 in.×4 in.×0.05 in.) was sprayed with 0.45 ml of 0.9% saline. 0.75 g of Degradable Starch Microspheres (DSM) was dusted over the section. The Surgicel®/saline/DSM composition was allowed to dry at room temperature overnight before drying at 55° C. for 48 hours.

1. A method for controlling bleeding at an active bleeding wound site of a mammal, the method comprising applying a hemostatic composition to the active bleeding wound site, the hemostatic composition comprising cellulose and a cross-linked polysaccharide ionically linked to the cellulose.

2. The method of claim 1, wherein the cross-linked polysaccharide is selected from covalently cross-linked dextran, covalently cross-linked starch, covalently cross-linked alginate, and ionically cross-linked alginate.

3. The method of claim 1, wherein the cross-linked polysaccharide is porous.

4. The method of claim 1, wherein the cross-linked polysaccharide has a molecular weight exclusion limit greater than about 10,000 Daltons when dry.

5. The method of claim 1, wherein the cross-linked polysaccharide has a molecular weight exclusion limit greater than about 30,000 Daltons when hydrated.

6. The method of claim 1, wherein the hemostatic composition is a bandage, suture, dressing, gauze, gel, foam, web, film, tape, or patch.

7. The method of claim 1, wherein the cross-linked polysaccharide is ionically linked to the cellulose via a cation selected from the group consisting of: K⁺, Na⁺, Mg²⁺, Ca²⁺, Ba²⁺, Zn²⁺, Cu²⁺, Fe³⁺, and Al³⁺.

8. The method of claim 7, wherein the cation is Na⁺.

9. The method of claim 7, wherein the counterion to said cation is selected from the group consisting of: F⁻, Cl⁻, Br⁻, I⁻, SO₄²⁻, PO₃³⁻, C₂H₃O⁻, C₆H₅O₇²⁻, C₄H₄O₆²⁻, C₂O₄², HCOO⁻, BO₃³⁻, and CO₃²⁻.

10. The method of claim 9, wherein the counterion is Cl⁻.

11. The method of claim 1, wherein the cellulose comprises cotton gauze.

12. The method of claim 1, wherein the covalently cross-linked polysaccharide is present at from about 0.5 g/104 cm² of cellulose to about 4 g/104 cm² of cellulose.

13. The method of claim 7, wherein the cation is present at from about 1×10⁻⁵ mols/cm² of cellulose to about 5×10⁻⁵ mols/cm² of cellulose.

14. The method of claim 1, wherein the hemostatic composition does not comprise alginate.

15. A hemostatic composition, comprising cellulose and Sephadex™ G-100, wherein the Sephadex™ G-100 is present from about 0.5 g/104 cm² of cellulose to about 4 g/104 cm² of cellulose, and wherein the Sephadex™ G-100 is ionically linked to the cellulose.

16. (canceled)

17. The hemostatic composition of claim 15, wherein the Sephadex™ G-100 is ionically linked to the cellulose via a cation selected from the group consisting of: K⁺, Na⁺, Mg²⁺, Ca²⁺, Ba²⁺, Zn²⁺, Cu²⁺, Fe³⁺, and Al³⁺.

18. The hemostatic composition of claim 17, wherein the cation is Na⁺.

19. The hemostatic composition of claim 15, wherein the cellulose is cotton gauze.

20. A method of making the hemostatic composition of claim 15, wherein the cellulose is contacted with a solution of a cation and contacted with Sephadex™ G-100.

21. A method for controlling bleeding at an arterial or venous wound of a mammal, the method comprising applying a hemostatic composition to the wounded artery or vein, the hemostatic composition comprising cellulose and a cross-linked polysaccharide ionically linked to the cellulose.

22. The method of claim 21, wherein the wound is located at the pulmonary artery or vein, aorta or vena cava, carotid artery or jugular vein, subclavian artery or vein, axillary artery or vein, brachial artery or vein, thoracic artery or vein, radial artery or vein, ulnar artery or vein, iliac artery or vein, femoral artery or vein, popliteal artery or vein, or tibial artery or vein.

23. The method of claim 21, wherein the cellulose is cotton gauze.

24. The method of claim 21, wherein the cross-linked polysaccharide is covalently cross-linked dextran beads.

25. The hemostatic composition of claim 15, wherein the Sephadex™ G-100 is present at about 2 g/104 cm² of cellulose.

26. The hemostatic composition of claim 15, wherein the Sephadex™ G-100 has a molecular weight exclusion limit greater than about 10,000 Daltons when dry.

27. The hemostatic composition of claim 15, wherein the Sephadex™ G-100 has a molecular weight exclusion limit greater than about 30,000 Daltons when hydrated.

28. The hemostatic composition of claim 15, wherein Sephadex™ G-100 is present at about 0.019 g per cm² of cellulose.
